(19) United States
(12) Patent Application Publication
(10) Pub. No.: US 2018/0055477 A1
(43) Pub. Date: Mar. 1, 2018

(54) Transcatheter Aortic Valve Implantation Assembly with Integrated Imager

(71) Applicant: Tyco Electronics Corporation, Berwyn, PA (US)

(72) Inventor: Alan D. Eskuri, Hanover, MN (US)

(73) Assignee: Tyco Electronics Corporation

(21) Appl. No.: 15/244,945

(22) Filed: Aug. 23, 2016

(51) Int. Cl. A61B 8/08; A61F 2/24; A61B 1/00; A61B 8/12; A61B 8/00; A61M 25/09

(52) U.S. Cl. CPC A61B 8/0841; A61F 2/2427; A61B 8/0891; A61B 1/00087; A61B 2034/2063; A61B 8/445; A61B 8/4494; A61M 25/09; A61B 8/0883; A61B 8/12

(57) ABSTRACT

A transcatheter aortic valve implantation (TAVI) delivery system includes a sheath, a replacement valve assembly disposed within a distal end of the sheath, and a nose cone proximate the distal end of the sheath. A forward section of the nose cone is configured to facilitate passage of the nose cone through the heart valve. The rear section of the nose cone comprises an imager.

### BACKGROUND

### Field

The present invention relates generally to a transcatheter aortic valve implantation assembly. More specifically, the present invention relates to transcatheter aortic valve implantation assembly with an integrated imager.

### Description of Related Art

Transcatheter aortic valve implantation, or TAVI for short, is a medical procedure in which a catheter is inserted in the artery in the leg and passed up into the heart to deliver a replacement valve assembly. The replacement valve assembly includes a metal stent and animal tissue integrated therein that performs the function of the valve to be replaced. The replacement valve assembly is typically positioned with a delivery system comprising a sheath and catheter prior to the procedure.

During the procedure, the delivery sheath is positioned within the damaged aortic valve. The delivery sheath is then pulled back to release the replacement valve assembly. Upon release, the stent expands within the damaged aortic valve thereby opening the valve and allowing the replacement valve integrated therein to fill the void and function as a valve.

Image devices outside of the patient such as x-ray devices or ultrasound devices may be utilized to provide internal images of the heart during the procedure. However, the images produced by such devices may be somewhat lacking due to image resolution, perspective and interference, which may impact the effectiveness of the procedure.

### SUMMARY

In one aspect, a transcatheter aortic valve implantation (TAVI) system includes a sheath, a replacement valve assembly disposed within a distal end of the sheath, and a nose cone proximate the distal end of the sheath. A forward section of the nose cone is configured to facilitate passage of the nose cone through the heart valve. The rear section of the nose cone comprises an imager.

In a second aspect, a nose cone for a transcatheter aortic valve implantation (TAVI) system includes a forward section configured to facilitate passage of the nose cone through the heart valve; and a rear section that comprises an imager.

### BRIEF DESCRIPTION OF THE DRAWINGS

FIG. 1A illustrates an exemplary embodiment of a transcatheter aortic valve implantation (TAVI) system as it passes through a damaged aortic valve;

FIG. 1B illustrates the TAVI system after a sheath of the TAVI system has been pulled back;

FIG. 2A illustrates a perspective view of a nose cone of the TAVI system; and

FIG. 2B illustrates a side view of the nose cone.

### DETAILED DESCRIPTION

To overcome the problems above, a transcatheter aortic valve implantation (TAVI) system with an integrated imager is disclosed below.

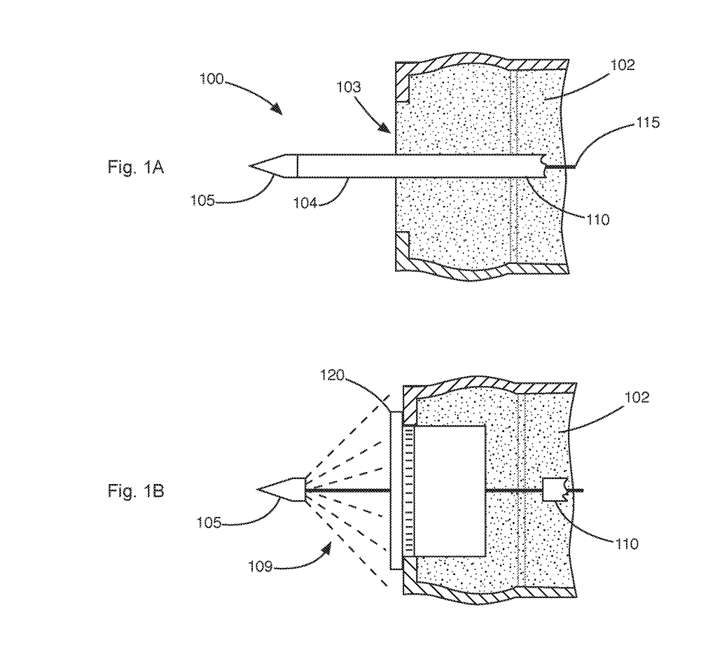

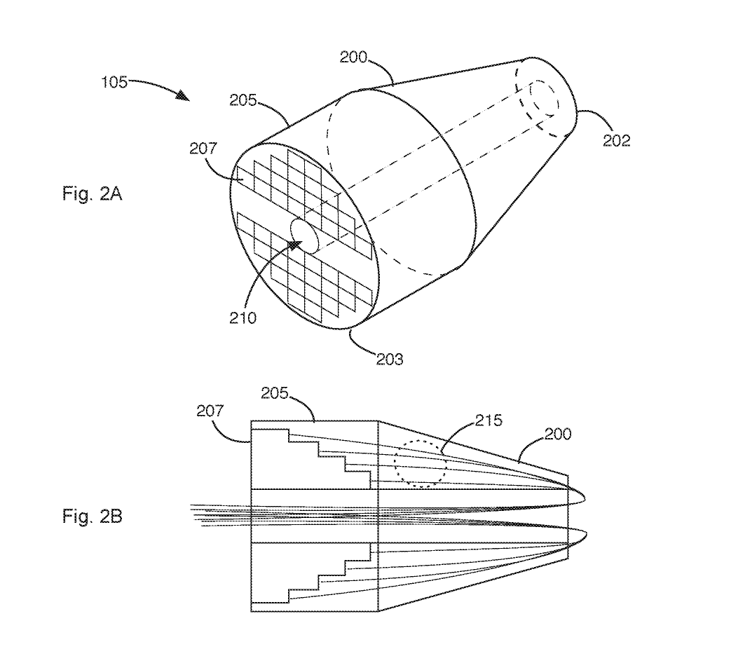

FIG. 1A illustrates an exemplary embodiment of a TAVI system 100 as it is passing through a damaged aortic valve 102. The TAVI system 100 includes a sheath 110 and a nose cone 105 that includes an integrated imager (205, FIG. 2A). In some implementations, the TAVI system 100 may include a guide wire 115 that extends through the sheath 110 and to the nose cone 105.

The sheath 110 may have a diameter of between about 18 Fr and 24 Fr. The sheath 110 may be formed from polyether block amide (PEBA), polyurethane, polyethylene, nylon, polyester, or other material suitable for insertion into the human body and flexible enough to be fed to the aortic valve or other area of the body.

As illustrated, the TAVI system 100 is inserted such that the nose cone 105 extends past the damaged aortic valve 102, into the left ventricle, and the distal end 104 of the sheath 110 is arranged within the opening 103 defined by the valve 102.

Referring to FIG. 1B, the sheath 110 may then be pulled back to release a replacement valve 120 that is disposed within the distal end 104 of the sheath. As illustrated, the relative position of the nose cone 105 with respect to the damaged aortic valve 102 remains generally the same between FIGS. 1A and 1B.

FIGS. 2A and 2B illustrate more detailed views of the nose cone. Referring to the figures, the nose cone 105 includes a tip portion and an imager 205. The nose cone 105 may be formed from polyether block amide (PEBA), polyurethane, polyethylene, nylon, polyester, or other material suitable for insertion into the human body. A forward section of the nose cone 105 may be configured to facilitate passage of the nose cone through the damaged aortic valve 102. For example, the nose cone 105 may have a tip portion 200 at a distal end 202 that is tapered to ease the passage of the nose cone 105 through the damaged aortic valve 102.

The imager 205 may be arranged at the proximal end of the nose cone 105 and facilitates providing a view of the damaged aortic valve 102 from within the left ventricle and facilitates viewing placement of the replacement valve 120, as illustrated by the dashed view lines 109 in FIG. 1B. In this regard, the imager 205 may correspond to a backward-looking 2D array of transducer elements 207.

The imager 205 may be integrally formed with the tip portion 200, as illustrated in the figures. However, in alternative embodiments, the imager 205 may be provided separately from the nose cone 105 and be fitted to the nose cone 105 at a later time via an adhesive or a different manner.

Each of the transducer elements 207 may correspond to a piezoelectric transducer (PZT) element, a capacitive micro machined ultrasonic transducer (CMUT) element, a piezoelectric micro machined ultrasonic transducer (PMUT) element, or a different type of transducer element. Forty transducer elements 207 are illustrated in FIG. 2A. However, a different number of transducer elements 207 may be utilized depending on a diameter of the delivery system and level of image resolution desired.

While the transducer elements 207 are illustrated as being arranged in a pyramid-like pattern on the back side of the nose cone 105, the transducer elements 207 may be arranged differently to suit a given situation. For example, the transducer elements 207 may be arranged to form a ring or concentric rings on the back side of the nose code 105. Other arrangements are possible.

As illustrated in FIG. 2B, a center channel 210 that extends from the proximal end of the nose cone 105 to the distal end 202 of the nose cone 105 facilitates the passage of conductors 215 from a back side of the imager 205 to an opening in the proximal end 203 of the nose cone 105. The conductors 215 may communicate imager-related signals from the transducer elements 207 to imaging equipment (not shown). The number of conductors 215 running through the nose cone 105 may correspond generally to the number of transducer elements 207. For example, forty conductors, plus a ground wire, power wire, etc., may run through the center channel 210 of the nose cone 105.

After exiting the opening in the proximal end 203 of the nose cone 210, the conductors 215 may run through the sheath 110 of the TAVI system 100, out of the patient, and to the imaging equipment. For example, the conductors 215 may run alongside the guide wire 115 and/or be spirally wrapped around the guide wire 115 at a desired turns/inch ratio.

In alternative implementations, the conductors 215 may run along the outside surface of the sheath 110. For example, the conductors 215 may spiral around the outside surface of the sheath 110 to provide a desired turns/inch ratio. The conductors 215 may run in a generally straight direction along the outside surface of the sheath 110. Other configurations are possible.

In some implementations, the conductors 215 may be embedded within the sidewall material from which the sheath 110 is formed. For example, the conductors 215 may be embedded within the sheath 110 during an extrusion process for forming the sheath 110. Alternatively, a channel (not shown) for feeding the conductors may be formed in the sheath 110, and the conductors 215 may be fed through the channel in subsequent operations.

While the TAVI system 100 has been described with reference to certain embodiments, it will be understood by those skilled in the art that various changes may be made and equivalents may be substituted without departing from the spirit and scope of the claims of the application. Various modifications may be made to adapt a particular situation or material to the teachings disclosed above without departing from the scope of the claims. Therefore, the claims should not be construed as being limited to any one of the particular embodiments disclosed, but to any embodiments that fall within the scope of the claims.

What is claimed is:

1. A transcatheter aortic valve implantation (TAVI) system comprising:
a sheath;
a replacement valve assembly disposed within a distal end of the sheath; and
a nose cone proximate the distal end of the sheath, wherein a forward section of the nose cone is configured to facilitate passage of the nose cone through the heart valve, and the rear section of the nose cone comprises an imager.

2. The TAVI system of claim 1, wherein the imager is a backward facing imager that facilitates viewing of the heart valve after the nose cone has passed through the heart valve and the sheath is pulled back.

3. The TAVI system of claim 1, wherein the imager is an ultrasonic imaging device.

4. The TAVI system of claim 1, wherein the imager comprises a backward-looking 2D array of transducer elements.

5. The TAVI system of claim 4, wherein the transducer elements are piezoelectric transducer (PZT) elements, capacitive micro machined ultrasonic transducer (CMUT) elements or piezoelectric micro machined ultrasonic transducer (PMUT) elements.

6. The TAVI system according to claim 1, further comprising a guide wire that extends through the sheath and to the nose cone.

7. The TAVI delivery system according to claim 1, wherein the forward section of the nose cone is tapered.

8. A nose cone for a transcatheter aortic valve implantation (TAVI) system comprising:
a forward section configured to facilitate passage of the nose cone through the heart valve; and
a rear section that comprises an imager.

9. The nose cone of claim 8, wherein the imager is a backward facing imager that facilitates viewing of a heart valve when the nose cone passes through the heart valve.

10. The nose cone of claim 8, wherein the imager is an ultrasonic imaging device.

11. The nose cone of claim 8, wherein the imager comprises a backward-looking 2D array of transducer elements.

12. The nose cone of claim 11, wherein the transducers are piezoelectric transducer (PZT) elements, capacitive micro machined ultrasonic transducer (CMUT) elements or piezoelectric micro machined ultrasonic transducer (PMUT) elements.

13. The nose cone according to claim 8, wherein the nose cone is mounted to an end of a guide wire that extends through a sheath.

14. The nose cone according to claim 8, wherein the forward section of the nose cone is tapere
